(19) United States
(12) Patent Application Publication
(10) Pub. No.: US 2013/0225876 A1
(43) Pub. Date: Aug. 29, 2013

(54) Hydrogenation Catalyst Using Multiple Impregnations of an Active Metal Solution

(71) Applicants: Heiko Weiner, Pasadena, TX (US); Zhenhua Zhou, Houston, TX (US)

(72) Inventors: Heiko Weiner, Pasadena, TX (US); Zhenhua Zhou, Houston, TX (US)

(73) Assignee: Celanese International Corporation, Dallas, TX (US)

(21) Appl. No.: 13/408,029

(22) Filed: Feb. 29, 2012

(51) Int. Cl. C07C 29/149; B01J 23/835; B01J 23/89; B01J 21/08; B01J 21/06; B01J 23/63; B01J 21/04; B01J 21/02; B01J 37/08; B01J 23/644; B01J 23/652; B01J 23/656; B01J 23/62; B01J 23/648

(52) U.S. Cl. 568/885; 502/339; 502/325; 502/326; 502/242; 502/334; 502/207; 502/213; 502/310; 502/324; 502/328; 502/330; 502/331

(57) ABSTRACT

A process for producing a catalyst that results in improved yields and productivity to ethanol. The process involves the steps of preparing a solution comprising one or more precursors to an active metal and impregnating a first portion of the solution on a support to form a first impregnated support. The first impregnated support is calcined to form a first calcined support and a second portion of the solution is impregnated on the first calcined support. The catalyst is useful for hydrogenating alkanoic acids to ethanol.

### FIELD OF THE INVENTION

The present invention relates generally to processes for hydrogenating alkanoic acids, in particular acetic acid, to form alcohols and to novel catalysts for use in such processes.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulose materials, such as corn or sugar cane. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol, which is suitable for fuels or human consumption. In addition, fermentation of starchy or cellulose materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. The reduction of various carboxylic acids over metal oxides has been proposed by EP0175558 and U.S. Pat. No. 4,398,039. A summary of some of the developmental efforts for hydrogenation catalysts for conversion of various carboxylic acids is provided in Yokoyama, et al., “Carboxylic acids and derivatives” in: Fine Chemicals Through Heterogeneous Catalysis, 2001, 370-379.

U.S. Pat. No. 6,495,730 describes a process for hydrogenating carboxylic acid using a catalyst comprising activated carbon to support active metal species comprising ruthenium and tin. U.S. Pat. No. 6,204,417 describes another process for preparing aliphatic alcohols by hydrogenating aliphatic carboxylic acids or anhydrides or esters thereof or lactones in the presence of a catalyst comprising Pt and Re. U.S. Pat. No. 5,149,680 describes a process for the catalytic hydrogenation of carboxylic acids and their anhydrides to alcohols and/or esters in the presence of a catalyst containing a Group VIII metal, such as palladium, a metal capable of alloying with the Group VIII metal, and at least one of the metals rhenium, tungsten or molybdenum. U.S. Pat. No. 4,777,303 describes a process for the productions of alcohols by the hydrogenation of carboxylic acids in the presence of a catalyst that comprises a first component which is either molybdenum or tungsten and a second component which is a noble metal of Group VIII on a high surface area graphitized carbon. U.S. Pat. No. 4,804,791 describes another process for the production of alcohols by the hydrogenation of carboxylic acids in the presence of a catalyst comprising a noble metal of Group VIII and rhenium. U.S. Pat. No. 4,517,391 describes preparing ethanol by hydrogenating acetic acid under superatmospheric pressure and at elevated temperatures by a process wherein a predominantly cobalt-containing catalyst.

Improvements to hydrogenation catalysts are needed.

### SUMMARY OF THE INVENTION

In a first embodiment, the present invention is directed to a process for producing a catalyst comprises the steps of: preparing a solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium; impregnating a first portion of the solution on a support to form a first impregnated support; calcining the first impregnated support to form a first calcined support; impregnating a second portion of the solution on the first calcined support to form a second impregnated support; and calcining the second impregnated support to form the catalyst.

In a second embodiment, the present invention is directed to a process for producing a catalyst, the process comprises the steps of: modifying a support or a precursor to form a modified support; preparing a solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium; impregnating a first portion of the solution on the modified support to form a first impregnated support; calcining the first impregnated support to form a first calcined support; impregnating a second portion of the solution on the first calcined support to form a second impregnated support; and calcining the second impregnated support to form the catalyst.

In a third embodiment, the present invention is directed to a process for producing an alcohol, the process comprises the steps of: passing a gaseous stream comprising hydrogen and an alkanoic acid in the vapor phase over a hydrogenation catalyst, wherein the hydrogenation catalyst is produced by the process comprises the steps of: preparing a solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium; impregnating a first portion of the solution on a support to form a first impregnated support; calcining the first impregnated support to form a first calcined support; impregnating a second portion of the solution on the first calcined support to form a second impregnated support; and calcining the second impregnated support to form the catalyst.

In a fourth embodiment, the present invention is directed to a process for preparing a hydrogenation catalyst, the process comprises: providing a support; preparing a solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium; impregnating a first portion of the solution on the support to form a first impregnated support; calcining the first impregnated support to form a first calcined support; impregnating a second portion of the solution on the first calcined support to form a second impregnated support; and calcining the second impregnated support to form the catalyst.

In a fifth embodiment, the present invention is directed to a process for producing a catalyst. The process comprises the steps of preparing a first solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium; impregnating a portion of the first solution on a support to form a first impregnated support; calcining the first impregnated support to form a first calcined support; preparing a second solution comprising one or more precursors to an active metal, wherein the second solution comprises the same one or more precursors to the active metal as the first solution; impregnating a portion of the second solution on the first calcined support to form a second impregnated support; and calcining the second impregnated support to form the catalyst.

### DETAILED DESCRIPTION OF THE INVENTION

The present invention relates to a process for the multiple impregnation of a solution containing one or more active metals onto a catalyst support to form a hydrogenation catalyst. The solution may be impregnated in several portions in sequence. Each portion contains the same active metals. In one exemplary embodiment, the present invention relates to processes for producing hydrogenation catalysts comprising the steps of impregnating a first portion of an active metal solution on a support to form a first impregnated support, calcining the first impregnated support to form a first calcined support, impregnating a second portion of the active metal solution on the first dried support to form a second impregnated support, and calcining the second impregnated support to form the hydrogenation catalyst. Therefore, the same metals are impregnated onto a catalyst support twice through each portion of the solution. In some embodiment, additional portions may be added from the same solution as necessary. The multiple impregnation of a solution may improve the dispersion of the active metals on the catalyst leading to increase catalyst performance.

In one embodiment, a single solution of active metal is prepared and separate portions of the solution are used for multiple impregnations. Each portion preferably is an aliquot of the single solution of active metals. In preferred embodiments, the first portion is from 40% to 60% of the active metal solution, e.g., from 45% to 55%. The second portion may also be from 40% to 60% of the active metal solution, e.g., from 45% to 55%. In another embodiment, the first portion is substantially equal to the second portion; preferably, the first and second portions are 50% of the active metal solution.

Although it may be convenient to prepare a single solution and draw separate portions, it is also contemplated that two solutions that contain the same active metal solutions may be prepared and sequentially impregnated. Thus, the multiple impregnations may be achieved using these separate solutions.

The present invention also relates to the hydrogenation of alkanoic acids into their corresponding alcohol using a hydrogenation catalyst produced by sequential impregnation according to one embodiment of the present invention. Embodiments of the present invention beneficially may be used in industrial applications to produce ethanol on an economically feasible scale. One particular preferred reaction is to make ethanol from acetic acid. The hydrogenation reaction may be represented as follows:

HOAc+2H₂→EtOH+H₂O

Surprisingly and unexpectedly the catalysts of the present invention provide higher conversion of acetic acid and higher selectivities to ethanol due to the multiple impregnations.

The active metal solution preferably uses water soluble compound or water dispersible compound/complex that includes the active metals of interest. Depending on the active metal precursor employed, the use of a solvent, such as water, glacial acetic acid, a strong acid such as hydrochloric acid, nitric acid, or sulfuric acid, or an organic solvent, may be preferred. In one embodiment, one or more metals or precursors are impregnated on the support. For example, one or more active metals or precursors may be used to prepare an impregnating solution. The solution may be divided into a first portion and a second portion and each portion of the solution contains the one or more active metals or precursors.

Impregnation occurs by adding, optionally drop wise, each portion of the solution, preferably in suspension or solution, to the dry support. The resulting mixture may then be heated, e.g., optionally under vacuum, in order to remove the solvent. Additional drying and calcining may be performed, optionally with ramped heating to form the final catalyst composition. Upon heating and/or the application of vacuum, the metal(s) of the metal precursor(s) preferably decompose into their elemental (or oxide) form. In some cases, the completion of removal of the liquid carrier, e.g., water, may not take place until the catalyst is placed into use and calcined, e.g., subjected to the high temperatures encountered during operation. During the calcination step, or at least during the initial phase of use of the catalyst, such compounds are converted into a catalytically active form of the metal or a catalytically active oxide thereof.

Drying may occur, for example, at a temperature of from 50° C. to 300° C., e.g., from 100° C. to 200° C. or about 120° C., optionally for a period of from 1 to 24 hours, e.g., from 3 to 15 hours or from 6 to 12 hours. Calcining of the catalyst may occur, for example, at a temperature of from 250° C. to 800° C., e.g., from 300 to 700° C. or about 500° C., optionally for a period of from 1 to 12 hours, e.g., from 2 to 10 hours, from 4 to 8 hours or about 6 hours.

In one embodiment, after the first portion of the solution with the active metal precursor is added to the support followed by drying and calcining, and the resulting material is then impregnated with the second portion of the solution with the active metal precursor followed by an additional drying and calcining step to form the final catalyst composition. Optionally, additional active metal precursors may also be impregnated into the support.

Suitable metal precursors include, for example, metal halides, amine solubilized metal hydroxides, metal nitrates, metal oxalates, or complex metal salts. For example, suitable compounds for metal precursors for platinum, palladium, and tin include chloroplatinic acid, ammonium chloroplatinate, amine solubilized platinum hydroxide, platinum nitrate, platinum tetra ammonium nitrate, platinum chloride, platinum oxalate, palladium nitrate, palladium tetra ammonium nitrate, palladium chloride, palladium oxalate, sodium palladium chloride, sodium platinum chloride ammonium perrhenate, sodium perrhenate, potassium perrhenate, rhenium heptoxide, potassium stannate, sodium stannate, stannic chloride, stannous chloride, stannous nitrate, stannous oxalate and the like. Generally, both from the point of view of economics and environmental aspects, aqueous solutions of soluble compounds of platinum are preferred.

In most cases, the impregnation may be carried out using metal nitrate solutions. However, various other soluble salts, which upon calcination release metal ions, can also be used. Examples of other suitable metal salts for impregnation include, metal acids, such as perrhenic acid solution, metal oxalates, and the like. In those cases where substantially pure ethanol is to be produced, it is generally preferable to use the nitrogenous amine and/or nitrate based precursors.

The solution may comprise one or more active metals or precursors thereto. The active metal or precursor thereto may be selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium. Preferably, the active metal or precursor thereto is selected from the group consisting of tin, iron, copper, platinum, palladium, chromium, tungsten, molybdenum, and nickel. Preferably, the metals or precursors thereto are tin and platinum. In another embodiment, the metals or precursors thereto are tin and ruthenium. In another embodiment, the metals or precursors thereto are tin and rhodium. In another embodiment, the metals or precursors thereto are tin and cobalt. In another embodiment, the metals or precursors thereto are cobalt, tin and platinum.

The total weight of all metals present in the final catalyst is at least 0.5 wt. % based on the total weight of the catalyst, e.g., 1 wt. % or 2 wt. %. The individual amounts of the metals may vary when multiple active metals are used. For example, the amount of each metal may be present in an amount from 0.1 to 20 wt. %, e.g., from 0.1 to 10 wt. %, or from 0.1 to 5 wt. %. When two metals are used, the molar ratio of the first metal to the second metal may be from 5:1 to 1:5, e.g., from 4:1 to 1:4, or from 3:1 to 1:3, or from 3:2 to 2:3.

Optionally, the catalyst may further comprise one or more metals selected from the group consisting of rhodium, cobalt, cesium, palladium, gold, iridium, and ruthenium. The optional metals are preferably different from the first and second metal.

For purposes of the present specification, unless otherwise indicated, weight percent is based on the total weight the catalyst including metal and support. The metal(s) in the catalyst may be present in the form of one or more metal oxides. For purposes of determining the weight percent of the metal(s) in the catalyst, the weight of any oxygen that is bound to the metal is ignored.

The catalysts of the present invention may be on any suitable support. In one embodiment, the support may be an inorganic oxide. In one embodiment, the support may be selected from the group consisting of silica, alumina, titania, silica/alumina, calcium metasilicate, pyrogenic silica, high purity silica, zirconia, carbon, zeolites and mixtures thereof. Preferably, the support comprises silica. In preferred embodiments, the support is present in an amount from 25 wt. % to 99 wt. %, e.g., from 30 wt. % to 98 wt. % or from 35 wt. % to 95 wt. %.

The surface area of the silicaceous support material, e.g., silica, preferably is at least about 50 m²/g, e.g., at least about 100 m²/g, at least about 150 m²/g, at least about 200 m²/g or most preferably at least about 250 m²/g. In terms of ranges, the silicaceous support material preferably has a surface area of from 50 to 600 m²/g, e.g., from 100 to 500 m²/g or from 100 to 300 m²/g. High surface area silica, as used throughout the application, refers to silica having a surface area of at least about 250 m²/g. For purposes of the present specification, surface area refers to BET nitrogen surface area, meaning the surface area as determined by ASTM D6556-04, the entirety of which is incorporated herein by reference.

The silicaceous support material also preferably has an average pore diameter of from 5 to 100 nm, e.g., from 5 to 30 nm, from 5 to 25 nm or from about 5 to 10 nm, as determined by mercury intrusion porosimetry, and an average pore volume of from 0.5 to 2.0 cm³/g, e.g., from 0.7 to 1.5 cm³/g or from about 0.8 to 1.3 cm³/g, as determined by mercury intrusion porosimetry.

The morphology of the support material, and hence of the resulting catalyst composition, may vary widely. In some exemplary embodiments, the morphology of the support material and/or of the catalyst composition may be pellets, extrudates, spheres, spray dried microspheres, rings, pentarings, trilobes, quadrilobes, multi-lobal shapes, or flakes although cylindrical pellets are preferred. Preferably, the silicaceous support material has a morphology that allows for a packing density from 0.1 to 1.0 g/cm³, e.g., from 0.2 to 0.9 g/cm³ or from 0.3 to 0.8 g/cm³. In terms of size, the silica support material preferably has an average particle size, e.g., meaning the diameter for spherical particles or equivalent spherical diameter for non-spherical particles, from 0.01 to 1.0 cm, e.g., from 0.1 to 0.7 cm or from 0.2 to 0.5 cm. Since the one or more metal(s) that are disposed on or within the modified support are generally very small in size, they should not substantially impact the size of the overall catalyst particles. Thus, the above particle sizes generally apply to both the size of the modified supports as well as to the final catalyst particles.

A preferred silica support material is SS61138 High Surface Area (HSA) Silica Catalyst Carrier from Saint-Gobain NorPro. The Saint-Gobain NorPro SS61138 silica contains approximately 95 wt. % high surface area silica; a surface area of about 250 m²/g; a median pore diameter of about 12 nm; an average pore volume of about 1.0 cm³/g as measured by mercury intrusion porosimetry and a packing density of about 0.352 g/cm³.

A preferred silica/alumina support material is KA-160 (Sud Chemie) silica spheres having a nominal diameter of about 5 mm, a density of about 0.562 g/ml, in absorptivity of about 0.583 g H₂O/g support, a surface area of about 160 to 175 m²/g, and a pore volume of about 0.68 ml/g.

In one embodiment, before the metals are impregnated, it may be desired to impregnate the support material with a support modifier. For example, an aqueous suspension of the support modifier may be formed by adding the solid support modifier to deionized water, followed by the addition of colloidal support material thereto. The resulting mixture may be stirred and added to additional support material using, for example, incipient wetness techniques in which the support modifier is added to a support material having the same pore volume as the volume of the support modifier solution. Capillary action then draws the support modifier into the pores in the support material. The modified support can then be formed by drying and calcining to drive off water and any volatile components within the support modifier solution and depositing the support modifier on the support material. Drying may occur, for example, at a temperature of from 50° C. to 300° C., e.g., from 100° C. to 200° C. or about 120° C., optionally for a period of from 1 to 24 hours, e.g., from 3 to 15 hours or from 6 to 12 hours. Once formed, the modified supports may be shaped into particles having the desired size distribution, e.g., to form particles having an average particle size in the range of from 0.2 to 0.4 cm. The supports may be extruded, pelletized, tabletized, pressed, crushed or sieved to the desired size distribution. Any of the known methods to shape the support materials into desired size distribution can be employed. Calcining of the shaped modified support may occur, for example, at a temperature of from 250° C. to 800° C., e.g., from 300° C. to 700° C. or about 500° C., optionally for a period of from 1 to 12 hours, e.g., from 2 to 10 hours, from 4 to 8 hours or about 6 hours.

In preferred embodiments, the total weight of the support modifiers are present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst.

Support modifiers may adjust the acidity of the support. For example, the acid sites, e.g. Bronsted acid sites, on the support material may be adjusted by the support modifier to favor selectivity to ethanol during the hydrogenation of acetic acid. The acidity of the support material may be adjusted by reducing the number or reducing the availability of Bronsted acid sites on the support material. The support material may also be adjusted by having the support modifier change the pKa of the support material. Unless the context indicates otherwise, the acidity of a surface or the number of acid sites thereupon may be determined by the technique described in F. Delannay, Ed., “Characterization of Heterogeneous Catalysts”; Chapter III: Measurement of Acidity of Surfaces, p. 370-404; Marcel Dekker, Inc., N.Y. 1984, the entirety of which is incorporated herein by reference. In particular, the use of modified supports that adjusts the acidity of the support to make the support less acidic or more basic favors formation of ethanol over other hydrogenation products.

In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIII metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include those selected from the group consisting of WO₃, MoO₃, Fe₂O₃, Cr₂O₃, V₂O₅, MnO₂, CuO, Co₂O₃, and Bi₂O₃.

In another embodiment, the support modifier may be a basic modifier that has a low volatility or no volatility. Such basic modifiers, for example, may be selected from the group consisting of: (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

As will be appreciated by those of ordinary skill in the art, supports and support modifiers are selected such that the catalyst system is suitably active, selective and robust under the process conditions employed for the formation of ethanol.

One advantage of catalysts of the present invention is the stability or activity of the catalyst for producing ethanol. Accordingly, it can be appreciated that the catalysts of the present invention are fully capable of being used in commercial scale industrial applications for hydrogenation of acetic acid, particularly in the production of ethanol. In particular, it is possible to achieve such a degree of stability such that catalyst activity will have a rate of productivity decline that is less than 6% per 100 hours of catalyst usage, e.g., less than 3% per 100 hours or less than 1.5% per 100 hours. Preferably, the rate of productivity decline is determined once the catalyst has achieved steady-state conditions.

In one embodiment, when the catalyst support comprises high purity silica, with calcium metasilicate as a support modifier, the catalyst activity may extend or stabilize, the productivity and selectivity of the catalyst for prolonged periods extending into over one week, over two weeks, and even months, of commercially viable operation in the presence of acetic acid vapor at temperatures of 125° C. to 350° C. at space velocities of greater than 2500 hr⁻¹.

The step of hydrogenating acetic acid may use any suitable hydrogenation process for producing ethanol. The materials, catalysts, reaction conditions, and separation processes that may be used in the hydrogenation of acetic acid are described further below.

The raw materials, acetic acid and hydrogen, fed to the primary reactor used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

The acetic acid fed to the hydrogenation reactor may also comprise other carboxylic acids and anhydrides, as well as aldehyde and/or ketones, such as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the hydrogenation reactor without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

The reactor, in some embodiments, may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed as the reactor, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation in the reactor may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 1500 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1. Generally, the reactor may use an excess of hydrogen, while the secondary hydrogenation reactor may use a sufficient amount of hydrogen as necessary to hydrogenate the impurities. In one aspect, a portion of the excess hydrogen from the reactor is directed to the secondary reactor for hydrogenation. In some optional embodiments, the secondary reactor could be operated at a higher pressure than the hydrogenation reactor and a high pressure gas stream comprising hydrogen may be separated from the secondary reactor liquid product in an adiabatic pressure reduction vessel, and the gas stream could be directed to the hydrogenation reactor system.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

In particular, the hydrogenation of acetic acid may achieve favorable conversion of acetic acid and favorable selectivity and productivity to ethanol in the primary reactor. For purposes of the present invention, the term “conversion” refers to the amount of acetic acid in the feed that is converted to a compound other than acetic acid. Conversion is expressed as a mole percentage based on acetic acid in the feed. The conversion may be at least 30%, e.g., at least 40%, or at least 60%. Although catalysts that have high conversions are desirable, such as at least 60%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. In one embodiment, catalyst selectivity to ethanol is at least 60%, e.g., at least 70%, or at least 80%. Preferably, the selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

In various embodiments of the present invention, the crude ethanol product produced by the reactor, before any subsequent processing, such as purification and separation, will typically comprise unreacted acetic acid, ethanol and water. As used herein, the term “crude ethanol product” refers to any composition comprising from 5 to 70 wt. % ethanol and from 5 to 40 wt. % water. Exemplary compositional ranges for the crude ethanol product are provided in Table 1. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 0 to 20 | 1 to 12 | 3 to 10 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product may comprise acetic acid in an amount less than 20 wt. %, e.g., of less than 15 wt. %, less than 10 wt. % or less than 5 wt. %. In terms of ranges, the acetic acid concentration of Table 1 may range from 0.1 wt. % to 20 wt. %, e.g., 0.2 wt. % to 15 wt. %, from 0.5 wt. % to 10 wt. % or from 1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is greater than 75%, e.g., greater than 85% or greater than 90%.

An ethanol product may be recovered from the crude ethanol product produced by the reactor using the catalyst of the present invention. The ethanol product may be recovered using several different techniques.

The ethanol product may be an industrial grade ethanol comprising from 75 to 96 wt. % ethanol, e.g., from 80 to 96 wt. % or from 85 to 96 wt. % ethanol, based on the total weight of the ethanol product. In some embodiments, when further water separation is used, the ethanol product preferably contains ethanol in an amount that is greater than 97 wt. %, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including fuels, solvents, chemical feedstock, pharmaceutical products, cleansers, sanitizers, hydrogenation transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, ethyl benzene, aldehydes, butadiene, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in copending U.S. Pub. Nos. 2010/0030002 and 2010/0030001, the entire contents and disclosures of which are hereby incorporated by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Yin U.S. Pat. No. 3,130,007, the entireties of which are hereby incorporated herein by reference.

In order that the invention disclosed herein may be more efficiently understood, an example is provided below. It should be understood that these examples are for illustrative purposes only and is not to be construed as limiting the invention in any manner.

### EXAMPLES

Single impregnation and multi-step impregnation of platinum-tin catalysts were synthesized and compared. The single impregnation platinum-tin catalyst was prepared by first dissolving 9.78 g of ammonium oxalate hydrate in 5.5 mL, of deionized H₂O. Next, 0.22 g of solid tin(II) oxalate was added to the solution and dissolved with stirring. Separately, 1.02 g of platinum oxalate solution (10 wt. % Pt) was diluted to 5.5 mL, with deionized H₂O. Next the platinum oxalate solution was added to the solution of the tin oxalate, and the resulting mixture was stirred for 5 minutes at room temperature. The solution was then used to impregnate the SiO₂—CaSiO₃ catalyst support, following by drying under vacuum, and at 120° C. under flowing air. The dried composition was calcined at 350° C. for 6 hours under flowing air.

The multi-step impregnation of platinum-tin catalyst was prepared using a two-step impregnation for the addition of the active metals. First, 0.20 g of ammonium oxalate hydrate was dissolved in 5.5 mL, of deionized H₂O. Next, 0.12 g of solid tin(II) oxalate was added to the solution and dissolved with stirring. Separately, 0.55 g of platinum oxalate solution (10 wt. % Pt) was diluted to 5.5 mL with deionized H₂O. Next, the platinum oxalate solution was added to the solution of the tin oxalate, and the resulting mixture was stirred for 5 minutes at room temperature. The solution was then used to impregnate the catalyst support, following by drying under vacuum, and at 120° C. under flowing air. Separately, 0.17 g of ammonium oxalate hydrated was dissolved in 4.75 mL of deionized H₂O. Nest, 0.10 g of solid tin(II) oxalate was added to the solution and dissolved with stirring. Separately, 0.47 g of platinum oxalate solution (10 wt. % Pt) was diluted to 4.75 mL with deionized H₂O. Next, the platinum oxalate solution was added to the solution of the tin oxalate, and the resulting mixture was stirred for 5 minutes at room temperature. The solution was then used to impregnate the dried catalyst support, following by drying under vacuum, and at 120° C. under flowing air. The final, dried, twice impregnated composition was then calcined at 350° C. for 6 hours under flowing air.

Table 2 shows the catalytic activities for single impregnation and multiple impregnation of SiO₂—CaSiO₃(6 wt. %)-Pt(1 wt. %)-Sn(1.2 wt. %) catalysts.

TABLE 2
Catalytic Activity for
SiO₂—CaSiO₃(6 wt. %)—Pt(1 wt. %)—Sn(1.2 wt. %)

| | Acetic Acid | Selectivity | | | EtOH | EtOH |
|---|---|---|---|---|---|---|
| | Conversion | EtOH | EtAcO | AcH | Yield | Prod |
| Impregnation | (%) | (%) | (%) | (%) | (%) | (g/kg/h) |
| Single | 57 | 81 | 14 | 1 | 46 | 508 |
| Multiple | 71 | 81 | 15 | 1 | 58 | 619 |

As shown in Table 2, acetic acid conversion improves from 57% for single impregnation to 71% for multiple impregnations, although the selectivity for ethanol, ethyl acetate and acetaldehyde did not vary. Due to the higher conversion of acetic acid, the catalyst prepared by the multiple impregnations of the present invention resulted in an improved yield and productivity of ethanol.

While the invention has been described in detail, modifications within the spirit and scope of the invention will be readily apparent to those of skill in the art. In addition, it should be understood that aspects of the invention and portions of various embodiments and various features recited herein and/or in the appended claims may be combined or interchanged either in whole or in part. Furthermore, those of ordinary skill in the art will appreciate that the foregoing description is by way of example only, and is not intended to limit the invention.

We claim:

1. A process for producing a catalyst, the process comprising the steps of:
preparing a solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium;
impregnating a first portion of the solution on a support to form a first impregnated support;
calcining the first impregnated support to form a first calcined support;
impregnating a second portion of the solution on the first calcined support to form a second impregnated support; and
calcining the second impregnated support to form the catalyst.

2. The process of claim 1, wherein the first portion is from 40% to 60% of the solution.

3. The process of claim 1, wherein the second portion is from 40% to 60% of the solution.

4. The process of claim 1, wherein the first portion is substantially equal to the second portion.

5. The process of claim 1, wherein the one or more precursors to the active metal are selected from the group consisting of tin, iron, copper, platinum, palladium, chromium, tungsten, molybdenum, and nickel.

6. The process of claim 1, wherein the one or more precursors to the active metal comprise a precursor to tin and a precursor to platinum.

7. The process of claim 1, wherein the one or more precursors to the active metal comprise a precursor to tin and a precursor to ruthenium.

8. The process of claim 1, wherein the one or more precursors to the active metal comprise a precursor to tin and a precursor to rhodium.

9. The process of claim 1, wherein the one or more precursors to the active metal comprise a precursor to tin and a precursor to cobalt.

10. The process of claim 1, wherein the one or more precursors to the active metal comprise a precursor to cobalt, a precursor to tin and a precursor to platinum.

11. The process of claim 1, wherein the support comprises a support material selected from the group consisting of silica, alumina, titania, silica/alumina, calcium metasilicate, pyrogenic silica, high purity silica, zirconia, carbon, zeolites, and mixtures thereof.

12. The process of claim 1, impregnating a precursor to a support modifier on the support to form a modified support.

13. The process of claim 12, wherein the support modifier is selected from the group consisting of TiO₂, ZrO₂, Nb₂O₅, Ta₂O₅, Al₂O₃, B₂O₃, P₂O₅, and Sb₂O₃.

14. The process of claim 12, wherein the support modifier is selected from the group consisting of WO₃, MoO₃, Fe₂O₃, Cr₂O₃, V₂O₅, Nb₂O₅, MnO₂, CuO, Co₂O₃, and Bi₂O₃.

15. The process of claim 12, wherein the support modifier is selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, and (viii) Group IIIB metal metasilicates.

16. The process of claim 1, wherein the first calcination occurs at a temperature from 300° C. to 700° C.

17. The process of claim 1, wherein the second calcination occurs at a temperature from 300° C. to 700° C.

18. The process of claim 1, wherein the one or more precursors are selected from the group consisting of metal halides, amine solubilized metal hydroxides, metal nitrates, complex metal salts and metal oxalates.

19. A catalyst produced by the process of claim 1 for converting acetic acid to ethanol.

20. A process for producing a catalyst the process comprising the steps of:
modifying a support to form a modified support;
preparing a solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium;
impregnating a first portion of the solution on the modified support to form a first impregnated support;
calcining the first impregnated support to form a first calcined support;
impregnating a second portion of the solution on the first calcined support to form a second impregnated support; and
calcining the second impregnated support to form the catalyst.

21. A process for producing ethanol comprising the steps of:
passing a gaseous stream comprising hydrogen and an alkanoic acid in the vapor phase over a hydrogenation catalyst, wherein the hydrogenation catalyst is produced by the process comprising the steps of:
preparing a solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium;
impregnating a first portion of the solution on a support to form a first impregnated support;
calcining the first impregnated support to form a first calcined support;
impregnating a second portion of the solution on the first calcined support to form a second impregnated support; and
calcining the second impregnated support to form the catalyst.

22. The process of claim 21, wherein the alkanoic acid is acetic acid.

23. The process of claim 21, wherein the conversion of acetic acid to ethanol is at least 30%.

24. The process of claim 21, wherein the selectivity to ethanol is at least 60%.

25. The process of claim 21, wherein the support comprises a support material selected from the group consisting of silica, alumina, titania, silica/alumina, calcium metasilicate, pyrogenic silica, high purity silica, zirconia, carbon, zeolites, and mixtures thereof.

26. The process of claim 21, further comprising:
impregnating a support modifier or precursor thereto onto the support to form a modified support.

27. The process of claim 21, wherein the alkanoic acid is formed from methanol and carbon monoxide, wherein at least one of the methanol, the carbon monoxide, and hydrogen for the hydrogenating step is derived from syngas, and wherein the syngas is derived from a carbon source selected from the group consisting of natural gas, oil petroleum, coal, biomass, and combinations thereof.

28. A process for preparing a hydrogenation catalyst, the process comprising:
providing a support;
preparing a solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium;
impregnating a first portion of the solution on the support to form a first impregnated support;
calcining the first impregnated support to form a first calcined support;
impregnating a second portion of the solution on the first calcined support to form a second impregnated support; and
calcining the second impregnated support to form the catalyst.

29. A process for producing a catalyst, the process comprising the steps of:
preparing a first solution comprising one or more precursors to an active metal, wherein the active metal is selected from the group consisting of rhenium, tin, iron, copper, lanthanum, cerium, platinum, palladium, cobalt, nickel, rhodium, ruthenium, vanadium, tungsten, molybdenum, chromium, zinc, calcium, magnesium, potassium, titanium, manganese, gold, and iridium;
impregnating a portion of the first solution on a support to form a first impregnated support;
calcining the first impregnated support to form a first calcined support;
preparing a second solution comprising one or more precursors to an active metal, wherein the second solution comprises the same one or more precursors to the active metal as the first solution;
impregnating a portion of the second solution on the first calcined support to form a second impregnated support; and
calcining the second impregnated support to form the catalyst.
